(19) United States
(12) Patent Application Publication
(10) Pub. No.: US 2008/0139626 A1
(43) Pub. Date: Jun. 12, 2008

(54) Novel Method

(76) Inventor: Stephen F. Rittenhouse, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/908,156

(22) PCT Filed: Mar. 10, 2006
(86) PCT No.: PCT/US06/08699

§ 371 Date: Sep. 10, 2007

Related U.S. Application Data
(60) Provisional application No. 60/660,296, filed on Mar. 10, 2005.

(51) Int. Cl. A61K 31/44; A61P 31/04

(52) U.S. Cl. 514/349

(57) ABSTRACT

A method for the treatment or prophylaxis of an infection in a mammal, said infection caused by an anti-microbial resistant bacteria, comprising, administering, to the mammal a pleuromutilin derivative is disclosed.

The present invention relates to a novel compound and to its use in medical therapy, particularly antibacterial therapy.

Pleuromutilin, the compound of formula (A), is a naturally occurring antibiotic which has antimycoplasmal activity and modest antibacterial activity. It has been shown that the antimicrobial activity can be improved by replacing the glycolic ester moiety at position 14 by an R—X—CH₂CO₂— group, where R is an aliphatic or aromatic moiety ad X is O, S, or NR′ (H Egger and H Reinshagen, J Antibiotics, 1976, 29, 923). Tiamulin, the compound of formula (B), which is used as a veterinary antibiotic, is a derivative of this type (G Hogenauer in Antibiotics, Vol. V, part 1, ed. F E Hahn, Springer-Verlag, 1979, p. 344).

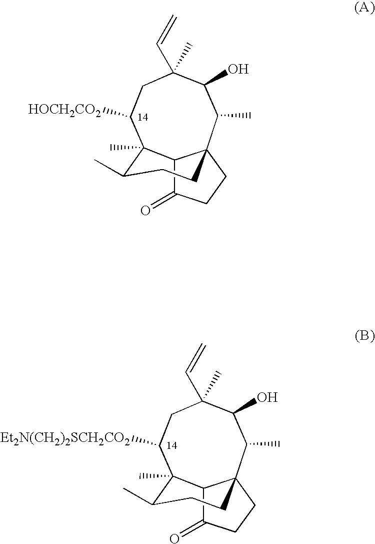

(In this application, the non-conventional numbering system which is generally used in the literature (G Hogenauer, loc.cit.) is used.)

WO 97/25309 (SmithKline Beecham) describes further modification of the acyloxy group, disclosing 14-O-carbamoyl derivatives of mutilin or 19, 20-dihydromutilin, in which the N-atom of the carbamoyl group is unsubstituted, mono- or di-substituted.

WO 98/05659 (SmithKline Beecham) discloses 14-O-carbamoyl derivatives of mutilin or 19,20-dihydromutilin, in which the N-atom of the carbamoyl group is acylated by a group which includes an azabicyclic moiety.

Many classes of antibacterial agents are known, including the penicillins and cephalosporins, tetracyclines, sulfonamides, monobactams, fluoroquinolones and quinolones, aminoglycosides, glycopeptides, macrolides, polymyxins, lincosamides, trimethoprim and chloramphenicol. The fundamental mechanisms of action of these antibacterial classes vary.

Bacterial resistance to many known antibacterials is a growing problem. Accordingly there is a continuing need in the art for alternative antibacterial agents.

Amongst the Gram-positive pathogens, such as staphylococci, streptococci, and enterococci, resistant strains have evolved/arisen which makes them particularly difficult to eradicate. Examples of such strains are methicillin resistant Staphylococcus aureus (MRSA), macrolide and quinolone resistant S. aureus, methicillin, macrolide and quinolone-resistant coagulase negative staphylococci, and penicillin, macrolide, quinolone-resistant Streptococcus pneumoniae.

In view of the rapid emergence of multidrug-resistant bacteria, the development of antibacterial agents with novel modes of action that are effective against the growing number of resistant bacteria, is of utmost importance.

WO 99/21855 (published Oct. 11, 2001) describes Compound A of the formula

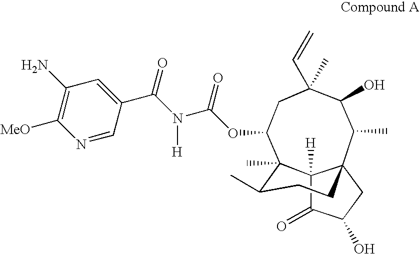

Applicants have now found that Compound A, its pharmaceutically acceptable salt, solvate, or physiologically functional derivative is highly effective against many anti-microbial resistant bacteria.

### SUMMARY OF THE INVENTION

In one aspect, the present invention includes a method for the treatment or prophylaxis of an infection in a mammal, said infection caused by anti-microbial resistant bacteria, comprising, administering to the mammal a therapeutically effective amount of Compound A or a salt, solvate, or physiologically functional derivative thereof.

Another aspect of the present invention includes pharmaceutical compositions comprising a therapeutically effective amount of Compound A, or a salt, solvate, or physiologically functional derivative thereof and one or more of pharmaceutically acceptable carriers, diluents and excipients for the treatment or prophylaxis of an infection in a mammal, said infection caused by anti-microbial resistant bacteria.

Another aspect of the present invention includes the use of Compound A, or a salt, solvate, or physiologically functional derivative thereof in the preparation of a medicament for use in the treatment of an infection in a mammal caused by anti-microbial resistant bacteria.

### BRIEF DESCRIPTION OF THE FIGURE

FIG. 1 describes in vivo microbiology of Compound A against S. aureus WCUH29 (MRSA).

### DETAILED DESCRIPTION

Preferred anti-microbial resistant organisms to be treated with Compound A are selected from the group consisting of:

S. aureus
MRSA: methicillin resistant Staphylococcus aureus
MupirocinR: mupirocin resistant
MRSA&MupR: methicillin resistant & mupirocin resistant
MacrolideR: macrolide resistant
KetolideR: keteolide resistant
QuinoloneR: quinolone resistant
TetracyclineR: tetracycline resistant
VRSA: vancomycin resistant Staphylococcus aureus
VISA: vancomycin intermediate (vanc. MIC 4 ug/ml) Staphylococcus aureus
LinezolidR: linezolid resistant
S. pneumoniae
PenicillinR: penicillin resistant
MacrolideR: macrolide resistant
QuinoloneR: quinolone resistant
H. influenzae
BLNAR beta lactamase negative ampicillin resistant
B-lactamase positive
ClarR: clarithromycin resistant

As used herein, the term “therapeutically effective amount” means any amount which, as, compared to a corresponding subject who has not received such amount, results in improved treatment, healing, prevention, or amelioration of an infection caused by anti-microbial resistant organisms.

As used herein, the term “physiologically functional derivative” refers to any pharmaceutically acceptable derivative of Compound A, for example, an ester or an amide, which upon administration to a mammal is capable of providing (directly or indirectly) Compound A or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute (in this invention, Compound A or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

The term “pharmaceutically acceptable salts” refer to non-toxic salts of Compound A. Salts of Compound A may comprise acid addition salts derived from the presence of a nitrogen. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate.

As used herein, the following terms have the meaning of

MRSA: methicillin resistant Staphylococcus aureus
MRSE: methicillin resistant Staphylococcus epidermidis
Mupirocin^{R}: mupirocin resistant
MRSA&Mup^{R}: methicillin resistant & mupirocin resistant Staphylococcus aureus
Macrolide^{R}: macrolide resistant
VRSA: vancomycin resistant Staphylococcus aureus
VISA (vanc. MIC 4): vancomycin intermediate Staphylococcus aureus with an MIC of 4 mcg/ml
Linezolid^{R}: Linezolid resistant
Ketek^{R}: Ketek (telithromycin) resistant
Meth^{R}/Ery^{R}: methicillin and erythromycin resistant
Meth^{R}: methicillin resistant
Pen^{R}: penicillin resistant
Eryth^{R}: erythromycin resistant
Cipro^{R}: ciprofloxacin resistant
BLNA^{R}: beta lactamase negative ampicillin resistant
Clar^{R}: clarithromycin resistant

While it is possible that, for use in therapy, therapeutically effective amounts of Compound A, as well as salts, solvates and physiological functional derivatives thereof, may be administered as the raw chemical, it is possible to present the active ingredient as a pharmaceutical composition. Accordingly, the invention further provides pharmaceutical compositions (otherwise referred to as pharmaceutical formulations), which include therapeutically effective amounts of Compound A and salts, solvates and physiological functional derivatives thereof, and one or more pharmaceutically acceptable carriers, diluents, or excipients. Compound A and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing Compound A, or salts, solvates and physiological functional derivatives thereof, with one or more pharmaceutically acceptable carriers, diluents or excipients.

Compound A may suitably be administered to the patient at a daily dosage of from 1.0 to 50 mg/kg of body weight. For an adult human (of approximately 70 kg body weight), from 50 to 3000 mg, for example about 1500 mg, of a compound according to the invention may be administered daily. Suitably, the dosage for adult humans is from 5 to 20 mg/kg per day. Higher or lower dosages may, however, be used in accordance with normal clinical practice.

More generally, Compound A may be formulated for administration in any convenient way for use in human or veterinary medicine, by analogy with other antibiotics. Preferred mammal to be treated with Compound A is a human.

Compound A may be formulated for administration by any route, for example oral, topical or parenteral. The compositions may, for example, be made up in the form of tablets, capsules, powders, granules, lozenges, creams, syrups, sprays or liquid preparations, for example solutions or suspensions, which may be formulated for oral use or in sterile form for parenteral administration by injection or infusion.

Tablets and capsules for oral administration may be in unit dosage form, and may contain conventional excipients including, for example, binding agents, for example, syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; and pharmaceutically acceptable wetting agents, for example sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or another suitable vehicle before use. Such liquid preparations may contain conventional additives, including, for example, suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate or acacia; non-aqueous vehicles (which may include edible oils), for example almond oil, oily esters (for example glycerine), propylene glycol, or ethyl alcohol; preservatives, for example methyl or propyl p-hydroxybenzoate or sorbic acid; and, if desired, conventional flavouring and colour agents.

Compositions according to the invention intended for topical administration may, for example, be in the form of ointments, creams, lotions, eye ointments, eye drops, ear drops, nose drops, nasal sprays, impregnated dressings, and aerosols, and may contain appropriate conventional additives, including, for example, preservatives, solvents to assist drug penetration, and emollients in ointments and creams. Such topical formulations may also contain compatible conventional carriers, for example cream or ointment bases, ethanol or oleyl alcohol for lotions and aqueous bases for sprays. Such carriers may constitute from about 1% to about 98% by weight of the formulation; more usually they will constitute up to about 80% by weight of the formulation.

Compositions according to the invention may be formulated as suppositories, which may contain conventional suppository bases, for example cocoa-butter or other glycerides.

Compositions according to the invention intended for parenteral administration may conveniently be in fluid unit dosage forms, which may be prepared utilizing the compound and a sterile vehicle, water being preferred. The compound, depending on the vehicle and concentration used, may be either suspended or dissolved in the vehicle. In preparing solutions, the compound may be dissolved in water for injection and filter-sterilised before being filled into a suitable vial or ampoule, which is then sealed. Advantageously, conventional additives including, for example, local anaesthetics, preservatives, and buffering agents can be dissolved in the vehicle. In order to enhance the stability of the solution, the composition may be frozen after being filled into the vial, and the water removed under vacuum; the resulting dry lyophilised powder may then be sealed in the vial and a accompanying vial of water for injection may be supplied to reconstitute the liquid prior to use. Parenteral suspensions may be prepared in substantially the same manner except that the compound is suspended in the vehicle instead of being dissolved and sterilisation cannot be accomplished by filtration. The compound may instead be sterilised by exposure to ethylene oxide before being suspended in the sterile vehicle. Advantageously, a surfactant or wetting agent is included in such suspensions in order to facilitate uniform distribution of the compound.

A composition according to the invention may suitably contain from 0.001% by weight, preferably (for other than spray compositions) from 10 to 60% by weight, of Compound A (based on the total weight of the composition), depending on the method of administration.

When the compositions according to the invention are presented in unit dosage form, for instance as a tablet, each unit dose may suitably comprise from 25 to 1000 mg, preferable from 50 to 500 mg, of Compound A.

### EXAMPLE

The following Examples illustrate the present invention and in no way limit the invention in any scope.

### Method to Determine MIC

Whole-cell antimicrobial activity was determined by broth microdilution. Test compounds were dissolved in DMSO and diluted 1:10 in water to produce a 64 mcg/ml stock solution. Using a 96 well microtitre plate, a Microlab AT Plus 2 (Hamilton Co., Reno, Nev.) serially diluted 50 ul of the stock solution into an appropriate broth medium. Staphylococcal isolates were tested using cation adjusted Mueller Hinton broth. For S. pneumonaie testing, the Mueller Hinton broth was supplemented with 5% lysed horse blood and H. influenzae was tested with Haemophilus Test Medium. After the compounds were diluted, a 50 ul aliquot of the test isolate (˜1×106 cfu/ml) prepared in normal saline was added to each well of the microtitre plate. The final test concentrations ranged from 0.016-16 ug/mL. Inoculated plates were incubated at 35° C. in ambient air for 18 to 24 hours. The minimum inhibitory concentration (MIC) was determined as the lowest concentration of compound that inhibited visible growth.

### MIC Data

TABLE 1
In vitro activity of Compoun A against Drug resistant organisms

| | | MIC (μg/mL) | | |
|---|---|---|---|---|
| Organism (phenotype) | N | Range | MIC₅₀ | MIC₉₀ |
| S. pneumoniae | 352 | | | |
| (penicillin-resistant) | | | | |
| Compound A | | 0.03-1 | 0.25 | 0.25 |
| Amox/clav | | 0.25-16 | 1 | 4 |
| Cefuroxime | | 1-32 | 4 | 8 |
| Azithromycin | | ≦0.015->16 | >16 | >16 |
| Telithromycin | | ≦0.002-16 | 0.25 | 2 |
| Levofloxacin | | 0.12->8 | 1 | 1 |
| Moxifloxacin | | 0.03->8 | 0.12 | 0.25 |
| S. pneumoniae | 552 | | | |
| (macrolide-resistant) | | | | |
| Compound A | | 0.015-1 | 0.25 | 0.25 |
| Amox/clav | | ≦0.015-16 | 1 | 4 |
| Cefuroxime | | ≦0.015-32 | 4 | 8 |
| Azithromycin | | 2->16 | >16 | >16 |
| Telithromycin | | ≦0.002-16 | 0.25 | 2 |
| Levofloxacin | | 0.12->8 | 1 | 2 |
| Moxifloxacin | | 0.015->8 | 0.12 | 0.25 |
| S. pneumoniae | 13 | | | |
| (levofloxacin-resistant) | | | | |
| Compound A | | 0.12-0.5 | 0.25 | 0.5 |
| Amox/clav | | ≦0.015-1 | 0.5 | 1 |
| Azithromycin | | 0.25->16 | 16 | >16 |
| Cefuroxime | | ≦0.015-8 | 2 | 8 |
| Levofloxacin | | 8->8 | >8 | >8 |
| Moxifloxacin | | 0.5->8 | 4 | 8 |
| Telithromycin | | ≦0.002-2 | 0.12 | 0.5 |
| S. aureus | 51 | | | |
| (methicillin-resistant) | | | | |
| Compound A | | 0.25-2 | 1 | 1 |
| Amox/clav | | 1->16 | 8 | 16 |
| Cefuroxime | | 0.25->32 | 4 | >32 |
| Azithromycin | | 2->16 | >16 | >16 |
| Telithromycin | | 0.12->16 | 0.25 | >16 |
| Levofloxacin | | 0.06->8 | 0.5 | >8 |
| Moxifloxacin | | 0.03->8 | 0.12 | 4 |
| S. pyogenes | 83 | | | |
| (macrolide-resistant) | | | | |
| Commpound A | | 0.12-0.25 | 0.25 | 0.25 |
| Amox/clav | | ≦0.015-0.12 | ≦0.015 | ≦0.015 |
| Cefuroxime | | ≦0.015-0.12 | ≦0.015 | ≦0.015 |
| Azithromycin | | 2->16 | 16 | >16 |
| Telithromycin | | 0.015->16 | 0.03 | 8 |
| Levofloxacin | | 0.12-2 | 0.5 | 1 |
| Moxifloxacin | | 0.06-0.5 | 0.12 | 0.25 |

TABLE 2
In vitro activity of Compound A against Drug resistant organisms

| | | MIC Range |
|---|---|---|
| Organism | | Compound |
| (# of isolates) | Resistance | (mcg/ml) |
| S. aureus (4) | VISA (vanc. MIC 4 μg/mL) | 0.5-4 |
| S. aureus (2) | VRSA (vanc MIC > 4 μg/mL) | 0.5 |
| S. aureus (3) | Linezolid^{R} | 2 |
| S. aureus (4) | Ketek^{R} | 0.5-4 |
| S. aureus (10) | Meth^{R}/Ery^{R} | 0.5-2 |
| S. aureus (6) | Tetracycline^{R} | 0.5-2 |
| S. aureus (8) | Mupirocin^{R} | 0.25-1 |
| S. epidermidis (6) | Meth^{R} | 0.25-1 |
| S. pneumoniae (5) | Pen^{R} | 0.06-0.25 |
| S. pneumoniae (5) | Eryth^{R} | 0.125-0.25 |
| S. pneumoniae (5) | Cipro^{R} | 0.125-0.25 |
| H. influenzae (5) | BLNAR | 2 |
| H. influenzae (5) | ClarR | 4-8 |

### In Vivo Efficacy

Oral efficacy of Compound A was evaluated in a murine groin abscess infection model caused by a methicillin resistant S. aureus WCUH29. Animals were infected via subcutaneous injection into the groin with 0.5 mL of inoculum (approximately 5.7 log₁₀ cfu/mouse). Groups of mice (n=5) received either Compound A (100, 300 or 600 mg/kg), moxifloxacin (100 mg/kg) or azithromycin (50 mg/kg).

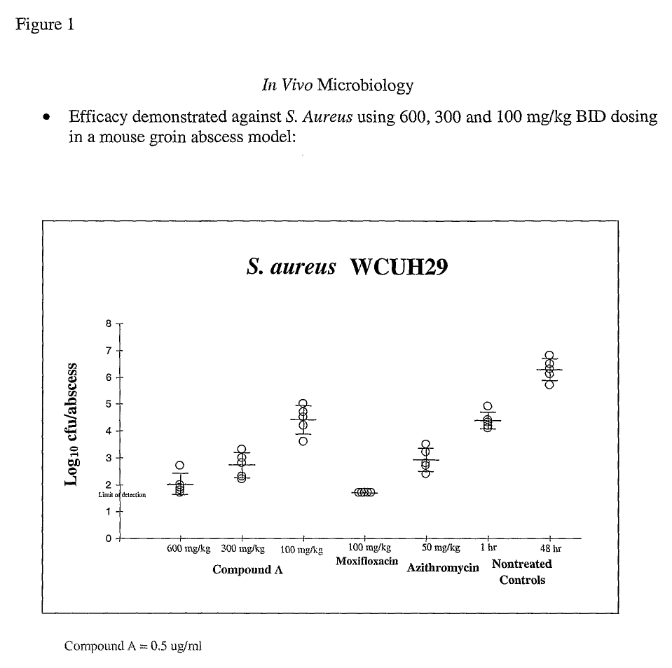

Compound A at 300 and 600 mg/kg was highly effective, reducing bacterial counts by 3.6 and 4.3 log₁₀ cfu/abscess, respectively (see FIG. 1) compared to non-treated controls (p≦0.01). The effect observed for Compound A at 600 mg/kg was comparable (p>0.05) to that observed for a 100 mg/kg dose of moxifloxacin (4.6 log₁₀ cfu/abscess reduction) and superior (p≦0.01) to the efficacy of a 50 mg/kg dose of azithromycin (3.4 log₁₀ cfu/abscess reduction).

What is claimed is:

1. A method for the treatment or prophylaxis of an infection in a mammal, said infection caused by an anti-microbial resistant bacteria, comprising, administering to the mammal a therapeutically effective amount of Compound A 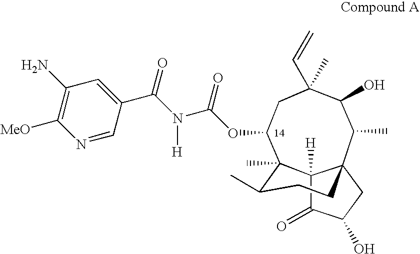
or a salt, solvate, or physiologically functional derivative thereof.

2. A pharmaceutical composition comprising a therapeutically effective amount of Compound A, or a salt, solvate, or physiologically functional derivative thereof and one or more of pharmaceutically acceptable carriers, diluents and excipients for the treatment or prophylaxis of an infection in a mammal, said infection caused by an anti-microbial resistant bacteria.

3. The method of claim 1 or the pharmaceutical composition of claim 2 in which the anti-microbial resistant bacteria is selected from the group consisting of:
S. aureus, S. pneumoniae and H. influenzae.

4. The method of claim 3 in which S. aureus is selected from the group consisting of:
MRSA, MupirocinR, MRSA&MupR, MacrolideR, KetolideR, QuinoloneR, TetracyclineR, VRSA, VISA (vanc. MIC 4), and LinezolidR.

5. The method of claim 3 in which S. pneumoniae is selected from the group:
PenicillinR, MacrolideR and QuinoloneR.

6. The method of claim 3 in which H. influenzae is selected from the group consisting of:
BLNAR, B-lactamase positive, and ClarR.

7. The pharmaceutical composition of claim 3 in which S. aureus is selected from the group consisting of:
MRSA, MupirocinR, MRSA&MupR, MacrolideR, KetolideR, QuinoloneR, TetracyclineR, VRSA, VISA (vanc. MIC 4), and LinezolidR.

8. The pharmaceutical composition of claim 3 in which S. pneumoniae is selected from the group:
PenicillinR, MacrolideR and QuinoloneR.

9. The pharmaceutical composition of claim 3 in which H. influenzae is selected from the group consisting of:
BLNAR, B-lactamase positive, and ClarR.
